(19) United States
(12) Patent Application Publication
(10) Pub. No.: US 2026/0191494 A1
(43) Pub. Date: Jul. 9, 2026

(54) APPLICATION OF SUPER-RESOLUTION IMAGING TECHNOLOGY IN VISUAL EVALUATION OF PANCREATIC B-CELL CHANGES

(71) Applicant: Zhejiang University, Hangzhou City (CN)

(72) Inventors: Pintong Huang, Hangzhou City (CN); Tao Zhang, Hangzhou City (CN); Jipeng Yan, Hangzhou City (CN); Zhan Shi, Hangzhou City (CN); Yiqing Zeng, Hangzhou City (CN); Xue Wang, Hangzhou City (CN); Guangrong Ma, Hangzhou City (CN)

(21) Appl. No.: 18/863,774

(22) PCT Filed: Jun. 12, 2023
(86) PCT No.: PCT/CN2023/099616

§ 371 Date: Nov. 7, 2024

(30) Foreign Application Priority Data
Apr. 24, 2023 (CN) 202310449145.9

(51) Int. Cl. A61B 8/06; A61B 8/00

(52) U.S. Cl. CPC A61B 8/06; A61B 8/481; A61B 8/5215; A61B 8/5276

(57) ABSTRACT

The present invention provides the application of super-resolution imaging technology in the visual evaluation of pancreatic β-cell changes within the field of vascular structure visualization. It utilizes super-resolution ultrasound technology to reconstruct the microvascular structure of the pancreas, enabling precise quantification of microvascular morphological and functional changes. This technology achieves accurate positioning of microbubbles at high concentrations, reducing the influence of low frame rates and displacement due to respiration and heartbeat during video acquisition. It enhances the accuracy of microvascular imaging and allows for precise quantification of microvascular morphological and functional changes, facilitating deeper medical research into pancreatic microvascular structure.

### BACKGROUND

The present invention relates to the application of super-resolution imaging technology in the visual evaluation of pancreatic β-cells changes within the field of visualization processing of vascular structures.

Super-resolution ultrasound imaging is the acoustic counterpart of optical super-resolution, offering spatial and temporal dimensions for longitudinal monitoring of disease progression. In super-resolution ultrasound imaging, microbubble signals in contrast-enhanced ultrasound images are localized and tracked across sequentially acquired frames to reconstruct the final super-resolution ultrasound image. This non-invasive imaging technique provides quantitative information about microvasculature and blood flow dynamics within its intact, actual physiological environment. It is being applied to imaging of the brain, tumors, and other microvasculature-related diseases.

### SUMMARY OF THE INVENTION

### Technical Issues

Type 2 diabetes is considered an autoimmune inflammatory condition. Prolonged chronic inflammation leads to pancreatic fibrosis, collagen deposition, and disruption of the structural and functional integrity of blood vessels, ultimately resulting in impaired β-cell function. The islets of Langerhans are highly vascularized structures, constituting roughly 10-20% of the pancreatic blood volume. Changes in islet blood flow occur during the progression of pancreatic inflammation and diabetes and in response to acute fluctuations in blood glucose, which are closely tied to overall pancreatic blood flow. However, existing in vivo imaging technologies, including functional MRI, Doppler ultrasound, and contrast-enhanced ultrasound, are limited in their ability to monitor the microvascular structures within the pancreas and the hemodynamics of deep tissues due to their resolution and sensitivity to slow flow within these vessels.

### Technical Solution

Given this, the present invention provides the application of super-resolution imaging technology in the visual evaluation of pancreatic β-cell changes to address the abovementioned issues in the prior art.

To achieve the above objectives, the present invention adopts the following technical solutions:

The application of super-resolution imaging technology in the visual evaluation of pancreatic β-cell changes includes the following steps:
S1. Optimize super-resolution imaging technology to achieve precise localization of microbubbles at lower frame rates and higher concentrations, enabling super-resolution ultrasound imaging based on clinical diagnostic equipment.
S2. Employ motion correction technology to overcome the influence of displacements such as respiration and heartbeat, thereby enhancing the accuracy of microvascular imaging.
S3. Visualize the distribution of organ microvessels and construct velocity maps, directional maps, etc., achieving micrometer-level resolution through super-resolution imaging technology.
S4. Utilize super-resolution imaging technology to accurately quantify parameters of microvascular morphology and function changes, including distortion, fractal dimension, blood flow velocity, vessel density, and vessel diameter.
S5. Perform three-dimensional reconstruction of microvascular pathological staining results from the model as the gold standard to verify the accuracy of super-resolution imaging technology in evaluating β-cell function and quality.

Furthermore, the steps for three-dimensional reconstruction of pathological staining results are as follows: Under anesthesia, 500 μL of FITC-conjugated tomato lectin is intravenously injected into the tail vein of rats. After pancreas isolation, the tissue is fixed in 4% PFA on ice for 1 hour and then cryopreserved overnight or until the tissue sinks in 30% sucrose. The lectin-injected pancreas is embedded in OCT medium, frozen in a cryomold, and sliced at 10-20 μm. Slices are imaged using an LSM800 confocal microscope (Zeiss) with excitation at 595 nm. Three-dimensional reconstruction is performed after combining 10 Z-axis confocal layers with a focusing step of 8-10 μm, resulting in a section thickness of ~Σ=80-100 μm.

### Beneficial Effects

The beneficial effects of the present invention lie in utilizing super-resolution ultrasound technology to reconstruct the microvascular structure of the pancreas, enabling precise quantification of microvascular morphological and functional changes. This technology achieves accurate microbubble localization at lower frame rates and higher concentrations, employing motion correction techniques to mitigate the effects of displacement due to respiration and heartbeat, thus enhancing the accuracy of microvascular imaging. Visualizing the distribution of organ microvessels and constricting blood flow velocity maps, directional maps, and others achieves micron-level resolution and precise quantification of microvascular morphological and functional changes. This facilitates deeper medical research into the pancreatic microvascular structure, aiming at evaluating β-cell function and quality.

### BRIEF DESCRIPTION OF THE FIGURES

FIG. 1 is a schematic diagram illustrating the natural progression of the T2D animal model developed in the present invention, aiming to simulate changes in β-cell quality and function.

FIG. 2 depicts a high-resolution ultrasound image of the microvasculature in the pancreas of rats.

FIG. 3 illustrates the calculation of resolution in super-resolution ultrasound imaging technology.

FIG. 4 presents the serological results of the T2D model at different stages of the disease progression.

### DETAILED DESCRIPTION OF THE INVENTION

### Best Mode of Carrying Out the Invention

Subsequently, with reference to the drawings in the embodiments of the present invention, the technical solutions in the embodiments of the present invention will be clearly and completely described. It is evident that the described embodiments are merely part of the embodiments of the present invention, not all of them. Based on the embodiments of the present invention, all other embodiments obtained by those skilled in the art without inventive labor are within the scope protected by the present invention.

### Embodiment

The steps for reconstructing pancreatic microvascular structure through super-resolution imaging are as follows:
S1. Optimize super-resolution imaging technology (with reference to the methods used in the paper “Super-Resolution Ultrasound Through Sparsity-Based Deconvolution and Multi-Feature Tracking”) to achieve precise microbubble localization at lower frame rates and higher concentrations, enabling super-resolution ultrasound imaging-based on clinical diagnostic equipment.
S2. Utilize motion correction techniques to overcome the influence of displacement caused by respiration and heartbeat, thereby enhancing the accuracy of microvascular imaging.
S3. Visualization of organ microvascular distribution through super-resolution imaging technology, facilitating the construction of flow velocity maps, directional maps, etc., achieving micron-level resolution;
S4. Employ super-resolution imaging technology to accurately quantify microvascular morphology and functional change parameters, including distortion, fractal dimension, blood flow velocity, vessel density, and vessel diameter.
S5. Under anesthesia, 500 μL of FITC-conjugated tomato lectin is intravenously injected into the tail vein of rats. After pancreas isolation, the tissue is fixed in 4% PFA on ice for 1 hour and then cryopreserved overnight or until the tissue sinks in 30% sucrose. The lectin-injected pancreas is embedded in OCT medium, frozen in a cryomold, and sliced at 10-20 μm thickness. Slices are imaged using an LSM800 confocal microscope (Zeiss) with excitation at 595 nm. Three-dimensional reconstruction is performed after combining 10 Z-axis confocal layers with a focusing step of 8-10 μm, resulting in a section thickness of ~Σ=80-100 μm. This served as the gold standard to validate the accuracy of super-resolution imaging techniques in assessing β-cell function and quality.

### Implementation of the Invention

### Embodiment 2

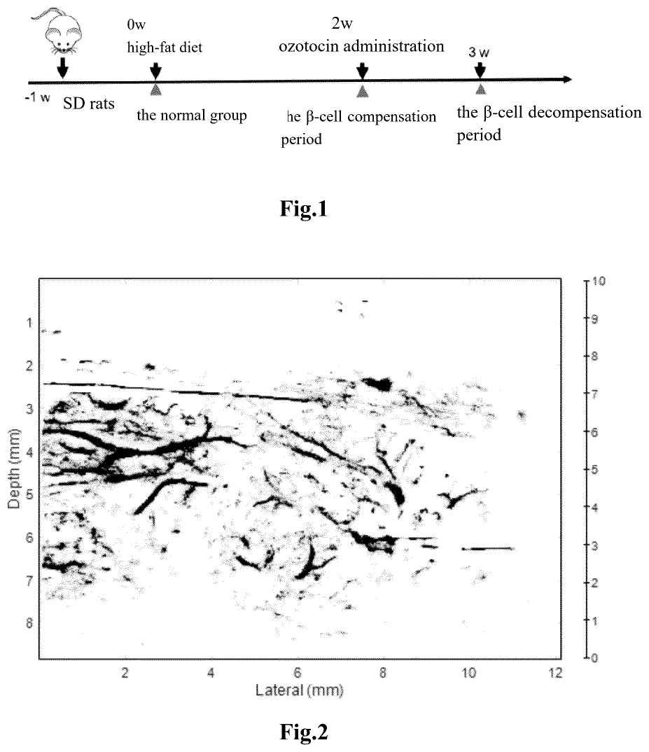

A rat model of type 2 diabetes (T2D) is established through a high-fat diet and streptozotocin (STZ) administration, as depicted in FIG. 1. In FIG. 1, following one week of adaptation feeding, two weeks of high-fat diet feeding, and the establishment of the insulin resistance model, STZ is injected intraperitoneally to induce the T2D model by the end of the third week, and the detection is carried out at the end of the fourth week.

The natural progression of changes in β-cell quality and function can be categorized into three stages: the normal group, the β-cell compensation period, and the β-cell decompensation period, as illustrated in FIG. 4. This modeling success is validated through intraperitoneal glucose tolerance tests (ipGTT), fasting serum insulin (FINS) levels, Homeostasis Model Assessment of Insulin Resistance (HOMA-IR), and Insulin Sensitivity Index (ISI).

Blood samples are collected from the posterior tibial artery, and blood glucose levels are measured using a fully automated biochemical analyzer. The serum is collected using tubes, then centrifuged at 1000 g for 10 minutes at 4° C. and used for biochemical analysis to detect the expression levels of insulin (SEKR-0023) for model evaluation.

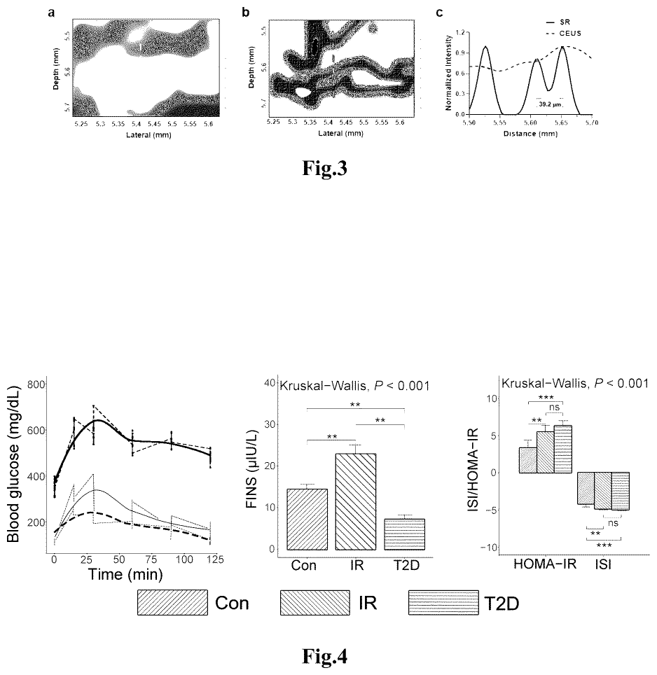

Conducting super-resolution imaging at different stages of the disease in Embodiment 1, quantifying various morphological and functional parameters of microvessels in different disease processes, as shown in FIG. 3. FIG. 3 illustrates that super-resolution imaging technology significantly improves the resolution of pancreatic microvessels, achieving a resolution of 39.2 μm, which is markedly higher than the vascular imaging obtained through maximum density projection. FIG. 2 depicts super-resolution imaging of microvessels in the pancreas of normal rats, demonstrating its ability to achieve a resolution of 39.2 μm.

1. The application of super-resolution imaging technology in visual evaluation of pancreatic β-cell changes includes the following steps:
S1. Optimize super-resolution imaging technology to achieve precise localization of microbubbles at lower frame rates and higher concentrations, enabling super-resolution ultrasound imaging based on clinical diagnostic equipment.
S2. Utilize motion correction techniques to overcome the influence of displacement caused by respiration and heartbeat, thereby enhancing the accuracy of microvascular imaging.
S3. Visualization of organ microvascular distribution through super-resolution imaging technology, facilitating the construction of flow velocity maps, directional maps, etc., achieving micron-level resolution;
S4. Employ super-resolution imaging technology to accurately quantify microvascular morphology and functional change parameters, including distortion, fractal dimension, blood flow velocity, vessel density, and vessel diameter.
S5. Perform three-dimensional reconstruction of microvascular pathological staining results from the model as the gold standard to verify the accuracy of super-resolution imaging technology in evaluating β-cell function and quality.

2. Furthermore, the steps for three-dimensional reconstruction of pathological staining results are as follows: Under anesthesia, 500 μL of FITC-conjugated tomato lectin is intravenously injected into the tail vein of rats. After pancreas isolation, the tissue is fixed in 4% PFA on ice for 1 hour and then cryopreserved overnight or until the tissue sinks in 30% sucrose. The lectin-injected pancreas is embedded in OCT medium, frozen in a cryomold, and sliced at 10-20 μm thickness. Slices are imaged using an LSM800 confocal microscope (Zeiss) with excitation at 595 nm. Three-dimensional reconstruction is performed after combining 10 Z-axis confocal layers with a focusing step of 8-10 μm, resulting in a section thickness of ~Σ=80-100 μm.
